(19) United States
(12) Patent Application Publication
(10) Pub. No.: US 2010/0272815 A1
(43) Pub. Date: Oct. 28, 2010

(54) AMORPHOUS FORM OF TAPENTADOL HYDROCHLORIDE

(75) Inventors: Mayur Devjibhai KHUNT, Gujarat (IN); Sandipan Prabhurao BONDGE, Maharashtra (IN); Vikas Daulatrao Ahirao, Maharashtra (IN); Nitin Sharadchandra Pradhan, Maharashtra (IN); Depashri Vikas Ahirao, Maharashtra (IN)

Correspondence Address: CANTOR COLBURN, LLP, 20 Church Street, 22nd Floor, Hartford, CT, 06103, US

(73) Assignee: ACTAVIS GROUP PTC EHF

(21) Appl. No.: 12/769,013

(22) Filed: Apr. 28, 2010

(30) Foreign Application Priority Data
Apr. 28, 2009 (IN) 977/CHE/2009

(51) Int. Cl. A61K 9/14; C07C 211/27; A61K 31/137; C07C 209/84; C07C 209/00; A61P 29/00; B32B 5/00

(52) U.S. Cl. 424/489; 564/381; 514/654; 428/402

(57) ABSTRACT

Disclosed herein is a novel and stable amorphous form of tapentadol hydrochloride, a process for the preparation, pharmaceutical compositions, and a method of treating thereof. Disclosed also herein is a stable amorphous co-precipitate of tapentadol hydrochloride with pharmaceutically acceptable excipients, a method for the preparation, pharmaceutical compositions, and a method of treating thereof. Advantageously, the amorphous co-precipitates of tapentadol hydrochloride have improved physiochemical characteristics that assist in the effective bioavailability.

### CROSS REFERENCE TO RELATED APPLICATION

This application claims the benefit of priority to Indian provisional application No. 977/CHE/2008, filed on Apr. 28, 2009, which is incorporated herein by reference in its entirety.

### FIELD OF THE INVENTION

Disclosed herein is a novel and stable amorphous form of tapentadol hydrochloride, process for the preparation, pharmaceutical compositions, and method of treating thereof. Disclosed also herein is a stable amorphous co-precipitates of tapentadol hydrochloride with pharmaceutically acceptable excipients, methods for the preparation, pharmaceutical compositions, and method of treating thereof.

### BACKGROUND

U.S. Reissue Pat. No. USRE39593 discloses a variety of 1-phenyl-3-dimethylaminopropane compounds, processes for their preparation, pharmaceutical compositions comprising the compounds, and method of use thereof. These compounds have the utility as analgesic active ingredients in pharmaceutical compositions. Among them, tapentadol hydrochloride, 3-[(1R,2R)-3-(dimethylamino)-1-ethyl-2-methylpropyl]phenol hydrochloride, is a centrally-acting analgesic with a unique dual mode of action as an agonist at the μ-opioid receptor and as a norepinephrine reuptake inhibitor. Tapentadol hydrochloride is represented by the following structural formula:

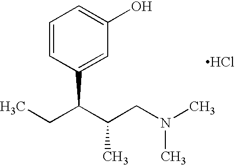

Various processes for the preparation of tapentadol, its enantiomers and related compounds, and their pharmaceutically acceptable salts are disclosed in U.S. Pat. Nos. 6,248,737 and 6,344,558; and PCT Publication Nos. WO 2004/108658, WO 2005/000788, WO 2008/012046, WO 2008/012047 and WO 2008/012283.

U.S. Patent Application No. 2007/0213405 (hereinafter referred to as the '405 application) discloses two crystalline polymorphs (Form A & Form B) of (−)-(1R,2R)-3-(3-dimethylamino-1-ethyl-2-methylpropyl)-phenol hydrochloride (tapentadol hydrochloride), and characterizes them by powder X-ray diffraction (P-XRD), Infra Red spectroscopy (IR), RAMAN spectroscopy and crystal structure analysis. The '405 application further teaches that the procedure described in example 25 of U.S. Pat. No. 6,248,737 and U.S. Pat. No. 6,344,558 as well as EP 693475 B1 produces crystalline Form B of tapentadol hydrochloride.

U.S. Patent Application No. 2009/0149534 (hereinafter referred to as the '534 application) discloses three crystalline modifications (modifications A, B & C) of tapentadol base, processes for their preparation, and characterizes the modifications by powder X-ray diffraction (P-XRD) pattern and RAMAN spectroscopy.

Polymorphism is defined as the ability of a substance to exist as two or more crystalline phases that have different arrangements and/or conformations of the molecule in the crystal lattice. Different polymorphs may differ in their physical properties such as melting point, solubility, X-ray diffraction patterns, and the like. Although these differences disappear once the compound is dissolved, they can appreciably influence the pharmaceutically relevant properties of the solid form, such as handling properties, dissolution rate and stability. Such properties can significantly influence the processing, shelf life, and commercial acceptance of a polymorph. It is therefore important to investigate all solid forms of a pharmaceutical compound, including all polymorphic forms, and to determine the stability, dissolution and flow properties of each polymorphic form.

Solvent medium and mode of isolation play very important roles in obtaining one polymorphic form over another.

An important solid state property of a pharmaceutical compound is its rate of dissolution in aqueous fluid. The rate of dissolution of an active ingredient in a patient's stomach fluid may have therapeutic consequences since it imposes an upper limit on the rate at which an orally-administered pharmaceutical compound may reach the patient's bloodstream. The rate of dissolution is a consideration in formulating syrups, elixirs and other liquid medicaments. The solid state form of a compound may also affect its behavior on compaction and its storage stability.

It has been disclosed in the art that the amorphous forms of a number of pharmaceutical compounds exhibit superior dissolution characteristics and in some cases different bioavailability patterns compared to crystalline forms [Konno T., Chem. Pharm. Bull., 38, 2003 (1990)]. For some therapeutic indications, one bioavailability pattern may be favored over another.

The discovery of new solid state forms of a pharmaceutical compound provides a new opportunity to improve the performance characteristics of a pharmaceutical product. It enlarges the repertoire of materials that a formulation scientist has available for designing, for example, a pharmaceutical dosage form of a pharmaceutical compound with a targeted release profile or other desired characteristic.

### SUMMARY OF THE INVENTION

The present inventors have now surprisingly and unexpectedly found a novel amorphous form of tapentadol hydrochloride and amorphous co-precipitates of tapentadol hydrochloride with pharmaceutically acceptable excipients that have high purity, adequate stability and good dissolution properties.

The novel amorphous form of tapentadol hydrochloride and its co-precipitates are consistently reproducible, do not have the tendency to convert to other forms, and are found to be more stable. The amorphous tapentadol hydrochloride and its co-precipitates exhibit properties making them suitable for formulating tapentadol hydrochloride.

In one aspect, encompassed herein is a process for preparing the highly pure and stable amorphous form of tapentadol hydrochloride.

In another aspect, the amorphous tapentadol hydrochloride has a water content of less than about 6% by weight, specifically less than about 5% by weight, and more specifically less than about 3% by weight, still more specifically less than about 1% by weight, and most specifically less than about 0.5% by weight, based on the total weight of the amorphous tapentadol hydrochloride.

In another aspect, provided herein is a pharmaceutical composition comprising amorphous tapentadol hydrochloride as disclosed herein and one or more pharmaceutically acceptable excipients.

In still another aspect, provided herein is a pharmaceutical composition comprising amorphous tapentadol hydrochloride made by the process disclosed herein, and one or more pharmaceutically acceptable excipients.

In still further aspect, encompassed herein is a process for preparing a pharmaceutical formulation comprising combining amorphous tapentadol hydrochloride with one or more pharmaceutically acceptable excipients.

In another aspect, provided herein are amorphous co-precipitates of tapentadol hydrochloride with pharmaceutically acceptable excipients. More particularly, disclosed herein are amorphous co-precipitates of tapentadol hydrochloride with improved physiochemical characteristics which help in the effective bioavailability of tapentadol hydrochloride. Such pharmaceutical compositions may be administered easily to a mammalian patient in a dosage form, e.g., liquid, powder, elixir, injectable solution, with a high rate of bioavailability.

In yet another aspect, encompassed herein is a process for preparing the novel and stable amorphous co-precipitates of tapentadol hydrochloride with pharmaceutically acceptable excipients.

The amorphous co-precipitate of tapentadol hydrochloride obtained by the processes described herein has improved solubility properties and hence also has improved bioavailability.

In another aspect, provided herein are pharmaceutical compositions comprising the amorphous co-precipitates of tapentadol hydrochloride and one or more pharmaceutically acceptable excipients.

In still further aspect, encompassed herein is a process for preparing pharmaceutical formulations comprising combining the amorphous co-precipitates of tapentadol hydrochloride with one or more pharmaceutically acceptable excipients.

In another aspect, the amorphous tapentadol hydrochloride or a co-precipitate thereof disclosed herein for use in the pharmaceutical compositions has a D₉₀ particle size of less than or equal to about 500 microns, specifically about 1 micron to about 300 microns, and most specifically about 10 microns to about 150 microns.

### BRIEF DESCRIPTION OF THE DRAWINGS

FIG. 1 is a characteristic powder X-ray diffraction (XRD) pattern of amorphous tapentadol hydrochloride.

FIG. 2 is a characteristic powder X-ray diffraction (XRD) pattern of an amorphous co-precipitate of tapentadol hydrochloride with povidone (1:1) prepared according to Example 4.

### DETAILED DESCRIPTION OF THE INVENTION

According to one aspect, there is provided a stable amorphous form of tapentadol hydrochloride or an amorphous co-precipitate comprising tapentadol hydrochloride and a pharmaceutically acceptable excipient. In one embodiment, the amorphous form of tapentadol hydrochloride and its co-precipitates disclosed herein contain less than about 10 percent crystalline forms of tapentadol hydrochloride, specifically less than 5 percent crystalline forms of tapentadol hydrochloride, more specifically less than 1 percent crystalline forms of tapentadol hydrochloride, and most specifically is essentially free of crystalline forms of tapentadol hydrochloride.

According to another aspect, there is provided a stable and substantially pure amorphous tapentadol hydrochloride having a water content of less than about 6% by weight, based on the total weight of the amorphous tapentadol hydrochloride.

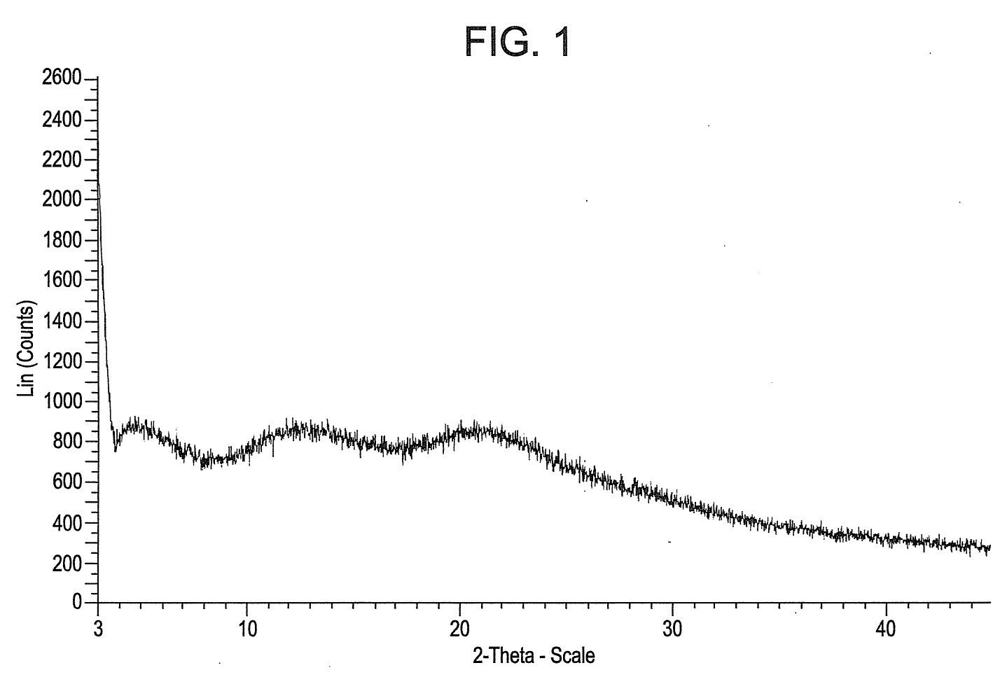

The amorphous form of tapentadol hydrochloride is characterized by a powder XRD pattern substantially in accordance with FIG. 1. The X-ray powder diffraction pattern shows no peaks, thus demonstrating the amorphous nature of the product.

According to another aspect, there is provided a process for the preparation of an amorphous form of tapentadol hydrochloride, comprising:

a) providing a solution of tapentadol hydrochloride in a solvent, wherein the solvent is water, an organic solvent, or a solvent medium comprising water and an organic solvent;

b) optionally, filtering the solution to remove insoluble matter;

c) optionally, seeding the solution; and

d) substantially removing the solvent from the solution to provide the amorphous form of tapentadol hydrochloride.

The term “substantially removing” the solvent refers to at least 80%, specifically greater than about 85%, more specifically greater than about 90%, still more specifically greater than about 99%, and most specifically essentially complete (100%), removal of the solvent from the solvent solution.

In one embodiment, the process produces an amorphous form of tapentadol hydrochloride in substantially pure form.

The term “substantially pure amorphous tapentadol hydrochloride” refers to the amorphous tapentadol hydrochloride having purity greater than about 99%, specifically greater than about 99.5%, more specifically greater than about 99.8% and still more specifically greater than about 99.9% (measured by HPLC).

In one embodiment, the amorphous tapentadol hydrochloride has a water content of less than about 6% by weight, specifically less than about 5% by weight, and more specifically less than about 3% by weight, still more specifically less than about 1% by weight, and most specifically less than about 0.5% by weight, based on the total weight of the amorphous tapentadol hydrochloride.

The amorphous tapentadol hydrochloride obtained by the process disclosed herein is stable, consistently reproducible, has good dissolution properties, and is particularly suitable for bulk preparation and handling. The amorphous tapentadol hydrochloride obtained by the process disclosed herein is suitable for formulating tapentadol hydrochloride.

Exemplary organic solvents used in step-(a) include, but are not limited to, an alcohol, a ketone, a chlorinated hydrocarbon, a hydrocarbon, a C₁-C₃ carboxylic acid, a nitrile, a polar aprotic solvent, and mixtures thereof. The term solvent also includes mixtures of solvents.

In one embodiment, the organic solvent is selected from the group consisting of methanol, ethanol, n-propanol, isopropyl alcohol, isobutanol, n-butanol, tert-butanol, acetone, methyl ethyl ketone, methyl isobutyl ketone, methyl tert-butyl ketone, methylene chloride, ethylene dichloride, chloroform, n-pentane, n-hexane, n-heptane, cyclohexane, toluene, xylene, acetic acid, acetonitrile, N,N-dimethylformamide, N,N-dimethylacetamide, dimethylsulfoxide, and mixtures thereof. Specifically, the organic solvent is selected from the group consisting of methanol, ethanol, isopropyl alcohol, acetone, methylene chloride, and mixtures thereof; and a most specific organic solvent is methanol.

Step-(a) of providing a solution of tapentadol hydrochloride includes dissolving tapentadol hydrochloride in the solvent, or obtaining an existing solution from a previous processing step.

In one embodiment, the tapentadol hydrochloride is dissolved in the solvent at a temperature of below about reflux temperature of the solvent used, specifically at about 20° C. to about 110° C., and still more specifically at about 25° C. to about 80° C.

As used herein, “reflux temperature” means the temperature at which the solvent or solvent system refluxes or boils at atmospheric pressure.

In another embodiment, the solution in step-(a) is prepared by admixing tapentadol base, hydrochloric acid and the solvent to obtain a mixture; and stirring the mixture to obtain a solution of tapentadol hydrochloride. In yet another embodiment, the mixture is stirred at a temperature of below about the reflux temperature of the solvent used for at least 15 minutes, specifically at about 20° C. to about 110° C. for about 20 minutes to about 10 hours, and still more specifically at about 25° C. to about 80° C. for about 30 minutes to about 2 hours.

Hydrochloric acid used may be in the form of aqueous hydrochloric acid, in the form of hydrogen chloride gas, or as hydrogen chloride dissolved in an organic solvent. The organic solvent used for dissolving hydrogen chloride gas or hydrogen chloride is selected from the group consisting of ethanol, methanol, isopropyl alcohol, ethyl acetate, diethyl ether, dimethyl ether and acetone

The solution obtained in step-(a) is optionally subjected to carbon treatment or silica gel treatment. The carbon treatment or silica gel treatment is carried out by methods known in the art, for example by stirring the solution with finely powdered carbon or silica gel at a temperature of below about 70° C. for at least 15 minutes, specifically at a temperature of about 40° C. to about 70° C. for at least 30 minutes; and filtering the resulting mixture through hyflo to obtain a filtrate containing tapentadol hydrochloride by removing charcoal or silica gel. Preferably, a finely powdered carbon is an active carbon. In one embodiment, a specific mesh size of silica gel is 40-500 mesh, and more specifically 60-120 mesh.

The solution obtained in step-(a) is optionally stirred at a temperature of about 30° C. to the reflux temperature of the solvent used for at least 20 minutes, and specifically at a temperature of about 40° C. to the reflux temperature of the solvent used from about 30 minutes to about 4 hours.

Removal of solvent in step-(d) is accomplished, for example, by substantially complete evaporation of the solvent, concentrating the solution, or distillation of solvent, under inert atmosphere to obtain amorphous tapentadol hydrochloride.

In one embodiment, the solvent is removed by evaporation. Evaporation can be achieved at sub-zero temperatures by lyophilisation or freeze-drying techniques. The solution may also be completely evaporated in, for example, a pilot plant Rota vapor, a Vacuum Paddle Dryer or in a conventional reactor under vacuum above about 720 mm Hg by flash evaporation techniques by using an agitated thin film dryer (“ATFD”), or evaporated by spray drying to obtain a dry amorphous powder.

The distillation process can be performed at atmospheric pressure or at reduced pressure. Specifically, the solvent is removed at a pressure of about 760 mm Hg or less, more specifically at about 400 mm Hg or less, still more specifically at about 80 mm Hg or less, and most specifically from about 30 to about 80 mm Hg.

Solvents can also be removed by spray-drying, in which a solution of tapentadol hydrochloride is sprayed into the spray drier at the flow rate ranging from 10 to 300 ml/hr, specifically 40 to 200 ml/hr. The air inlet temperature to the spray drier used may range from about 30° C. to about 150° C., specifically from about 65° C. to about 110° C. and the outlet air temperature used may range from about 30° C. to about 90° C.

Another suitable method is vertical agitated thin-film drying (or evaporation). Agitated thin film evaporation technology involves separating the volatile component using indirect heat transfer coupled with mechanical agitation of the flowing film under controlled conditions. In vertical agitated thin-film drying (or evaporation) (ATFD-V), the starting solution is fed from the top into a cylindrical space between a centered rotary agitator and an outside heating jacket. The rotor rotation agitates the downside-flowing solution while the heating jacket heats it.

The pure amorphous tapentadol hydrochloride obtained by above process may be further dried in, for example, a Vacuum Tray Dryer, a Rotocon Vacuum Dryer, a Vacuum Paddle Dryer or a pilot plant Rota vapor, to further lower residual solvents. Drying can be carried out under reduced pressure until the residual solvent content reduces to the desired amount such as an amount that is within the limits given by the International Conference on Harmonization of Technical Requirements for Registration of Pharmaceuticals for Human Use (“ICH”) guidelines.

In one embodiment, the drying is carried out at atmospheric pressure or reduced pressures, such as below about 200 mm Hg, or below about 50 mm Hg, at temperatures such as about 25° C. to about 70° C. The drying can be carried out for any desired time period that achieves the desired result, such as times about 1 to 20 hours. Drying may also be carried out for shorter or longer periods of time depending on the product specifications. Temperatures and pressures will be chosen based on the volatility of the solvent being used and the foregoing conditions should be considered as only a general guidance. Drying can be suitably carried out in a tray dryer, vacuum oven, air oven, or using a fluidized bed drier, spin flash dryer, flash dryer, and the like. Drying equipment selection is well within the ordinary skill in the art.

The total purity, including the chemical and enantiomeric purity, of the amorphous tapentadol hydrochloride obtained by the process disclosed herein is greater than about 99%, specifically greater than about 99.5%, more specifically greater than about 99.9%, and most specifically greater than about 99.95% as measured by HPLC. For example, the purity of the amorphous tapentadol hydrochloride can be about 99% to about 99.95%, or about 99.5% to about 99.99%.

According to another aspect, there are provided amorphous co-precipitates comprising tapentadol hydrochloride and a pharmaceutically acceptable excipient, having improved physiochemical characteristics that assist in the effective bioavailability of tapentadol hydrochloride. In one embodiment, the pharmaceutically acceptable excipient is selected from the group consisting of polyvinylpyrrolidone (also called povidone), polyvinyl alcohol, hydroxypropyl methylcellulose, methyl cellulose, carboxymethyl cellulose, sodium carboxymethyl cellulose, hydroxyethylcellulose, polyvinyl acetate, maltodextrins, cyclo dextrins, gelatins, hypromellose phthalate, sugars, and combinations comprising one or more of the foregoing hydrophilic carriers. A specific pharmaceutically acceptable excipient is povidone.

According to another aspect, there are provided pharmaceutical compositions comprising amorphous co-precipitates of tapentadol hydrochloride, and one or more pharmaceutically acceptable excipients.

The amorphous co-precipitates of tapentadol hydrochloride with a pharmaceutically acceptable carrier obtained by the processes disclosed herein are characterized by one or more of their powder X-ray diffraction (XRD) pattern, infrared absorption (IR) spectrum, and SEM images of the morphological analysis.

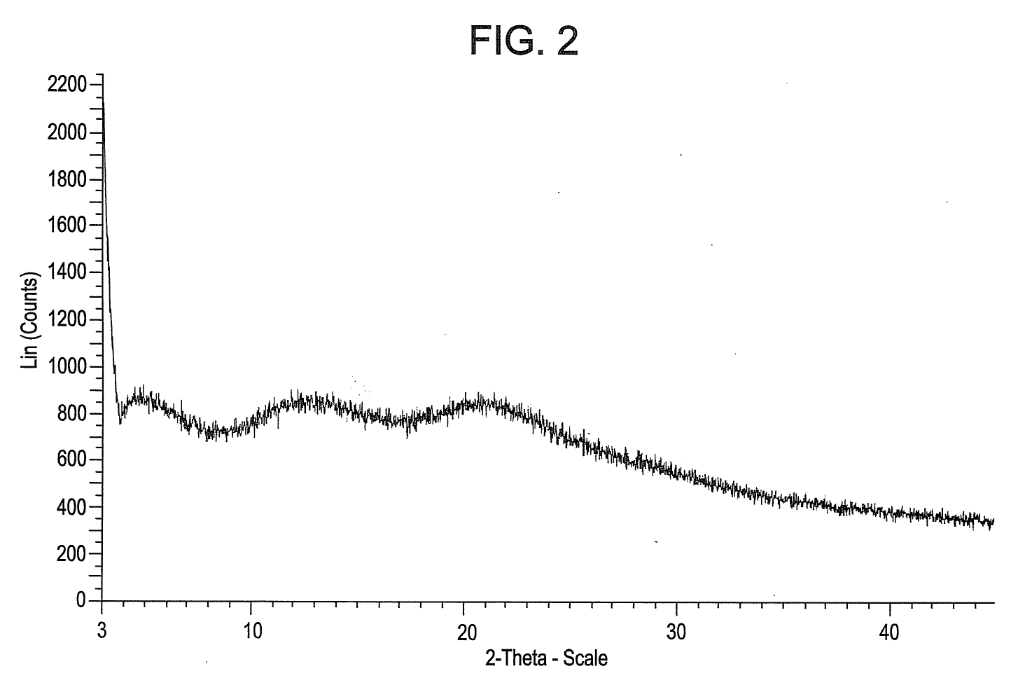

According to another aspect, there is provided an amorphous co-precipitate of tapentadol hydrochloride with povidone, characterized by a powder X-ray diffraction pattern, showing a plain halo with no well-defined peaks, substantially in accordance with FIG. 2.

In one embodiment, the amorphous co-precipitate of tapentadol hydrochloride with povidone disclosed herein remains in the same amorphous form and is stable when stored at a temperature of about 25±2° C. and at a relative humidity of about 55±5% for a period of at least 3 months.

In another embodiment, the amorphous co-precipitate of tapentadol hydrochloride with povidone disclosed herein remains in the same amorphous form and is stable when stored at a temperature of about 25±2° C. and at a relative humidity of about 55±5% for a period of 12 months.

The term “remains stable”, as defined herein, refers to lack of formation of impurities, while being stored as described hereinbefore.

According to another aspect, there is provided a process for preparing an amorphous co-precipitate of tapentadol hydrochloride and a pharmaceutically acceptable excipient, comprising:

a) providing a solution of tapentadol hydrochloride and a pharmaceutically acceptable excipient in a solvent, wherein the solvent is water, an organic solvent, or a solvent medium comprising water and an organic solvent;

b) optionally, filtering the solvent solution to remove insoluble matter; and

c) substantially removing the solvent from the solution to afford the amorphous co-precipitate of tapentadol hydrochloride with the pharmaceutically acceptable excipient.

The process can produce amorphous co-precipitates of tapentadol hydrochloride with a pharmaceutically acceptable excipient in substantially pure form.

The amorphous co-precipitates of tapentadol hydrochloride obtained by the process disclosed herein are stable, consistently reproducible and have good flow properties, and which is particularly suitable for bulk preparation and handling. The novel co-precipitates obtained by the process disclosed herein are suitable for formulating tapentadol hydrochloride.

In one embodiment, the pharmaceutically acceptable excipient used in step-(a) is selected from the group as described above. A specific pharmaceutically acceptable excipient is povidone. The use of mixtures of more than one of the pharmaceutical carriers to provide desired release profiles or for the enhancement of stability is within the scope of this invention. Also, all viscosity grades, molecular weights, commercially available products, their copolymers, mixtures are all within the scope of this invention without limitation.

In one embodiment, the povidone may be chosen from one or more of the grades such as PVP K-15, K-25, K-30, K29/32, K-60 and K-90.

The organic solvent used in step-(a) is selected from the group consisting of solvents described herein above. A most specific organic solvent is methanol.

Step-(a) of providing a solution of tapentadol hydrochloride includes dissolving a form of tapentadol hydrochloride in the solvent, or such a solution may be obtained directly from a reaction in which tapentadol hydrochloride is formed. The pharmaceutical excipient can be dissolved in a solution containing tapentadol hydrochloride, or, tapentadol hydrochloride can be dissolved in a solution containing a pharmaceutical excipient.

Alternatively, a solution containing tapentadol hydrochloride can be combined with a solution containing a pharmaceutically acceptable excipient, and the solvents used for preparing the different solutions need not be the same as long as the solvents have mutual solubility and form a single phase. In any event, tapentadol hydrochloride should be completely soluble in the solvents used and should provide a clear solution. The presence of undissolved crystals could lead to the formation of a material that is not completely amorphous.

In one embodiment, the dissolution is carried out at a temperature of about 0° C. to about 140° C., specifically at about 20° C. to about 100° C., and more specifically at about 25° C. to about 80° C.

In another embodiment, the solution obtained in step-(a) is optionally be subjected to carbon treatment or silica gel treatment according to the methods described herein above.

The solution obtained in step-(a) is optionally stirred at a temperature of about 30° C. to the reflux temperature of the solvent used for at least 20 minutes, and specifically at a temperature of about 40° C. to the reflux temperature of the solvent used for about 30 minutes to about 4 hours.

Removal of solvent in step-(c) is accomplished by the methods described herein above. The amorphous co-precipitates of tapentadol hydrochloride with pharmaceutically acceptable excipient obtained by the above process may be further dried according to the methods described herein above.

The dried product obtained by the process disclosed herein above can optionally be milled to get desired particle sizes. Milling or micronization can be performed prior to drying, or after the completion of drying of the product. The milling operation reduces the size of particles and increases surface area of particles. Drying is more efficient when the particle size of the material is smaller and the surface area is higher, hence milling will frequently be performed prior to the drying operation.

Milling can be done suitably using jet milling equipment like an air jet mill, or using other conventional milling equipment.

The resulting amorphous powder compositions disclosed herein have improved solubility properties and hence also have improved bioavailability.

The amorphous co-precipitates of tapentadol hydrochloride with the pharmaceutically acceptable excipients obtained by the process disclosed herein are a random distribution of the tapentadol hydrochloride and the pharmaceutically acceptable excipient in a particle matrix. Without being held to any particular theory, the co-precipitates have the characteristics of solid dispersions at a molecular level, being in the nature of solid solutions. The solid solutions, or molecular dispersions, provide homogeneous particles in which substantially no discrete areas of only amorphous tapentadol hydrochloride and/or only pharmaceutically acceptable excipient can be observed.

Further encompassed herein is the use of the amorphous form of tapentadol hydrochloride and co-precipitates thereof for the manufacture of a pharmaceutical composition together with a pharmaceutically acceptable carrier.

A specific pharmaceutical composition of the amorphous tapentadol hydrochloride is selected from a solid dosage form and an oral suspension.

In one embodiment, the amorphous form of tapentadol hydrochloride or a co-precipitate thereof has a D₉₀ particle size of less than or equal to about 500 microns, specifically about 1 micron to about 300 microns, and most specifically about 10 microns to about 150 microns.

In another embodiment, the substantially pure amorphous form of tapentadol hydrochloride or a co-precipitate thereof disclosed herein for use in the pharmaceutical compositions has a D₉₀ particle size of less than or equal to about 500 microns, specifically about 1 micron to about 300 microns, and most specifically about 10 microns to about 150 microns.

In another embodiment, the particle sizes of the amorphous form of tapentadol hydrochloride or a co-precipitate thereof can be achieved by a mechanical process of reducing the size of particles which includes any one or more of cutting, chipping, crushing, milling, grinding, micronizing, trituration or other particle size reduction methods known in the art, to bring the solid state form to the desired particle size range.

According to another aspect, there is provided a method for method for treating a patient suffering from severe acute pain, comprising administering a therapeutically effective amount of the amorphous tapentadol hydrochloride or a co-precipitate thereof, or a pharmaceutical composition that comprises a therapeutically effective amount of amorphous tapentadol hydrochloride or a co-precipitate thereof, along with pharmaceutically acceptable excipients.

According to another aspect, there are provided pharmaceutical compositions comprising amorphous tapentadol hydrochloride or a co-precipitate thereof prepared according to processes disclosed herein and one or more pharmaceutically acceptable excipients.

According to another aspect, there is provided a process for preparing a pharmaceutical formulation comprising combining amorphous tapentadol hydrochloride or a co-precipitate thereof prepared according to processes disclosed herein, with one or more pharmaceutically acceptable excipients.

Yet in another embodiment, pharmaceutical compositions comprise at least a therapeutically effective amount of amorphous tapentadol hydrochloride or a co-precipitate thereof. Such pharmaceutical compositions may be administered to a mammalian patient in a dosage form, e.g., solid, liquid, powder, elixir, aerosol, syrups, injectable solution, etc. Dosage forms may be adapted for administration to the patient by oral, buccal, parenteral, ophthalmic, rectal and transdermal routes or any other acceptable route of administration. Oral dosage forms include, but are not limited to, tablets, pills, capsules, syrup, troches, sachets, suspensions, powders, lozenges, elixirs and the like. The amorphous tapentadol hydrochloride or a co-precipitate thereof may also be administered as suppositories, ophthalmic ointments and suspensions, and parenteral suspensions, which are administered by other routes.

The pharmaceutical compositions further contain one or more pharmaceutically acceptable excipients. Suitable excipients and the amounts to use may be readily determined by the formulation scientist based upon experience and consideration of standard procedures and reference works in the field, e.g., the buffering agents, sweetening agents, binders, diluents, fillers, lubricants, wetting agents and disintegrants described hereinabove.

In one embodiment, capsule dosage forms contain amorphous tapentadol hydrochloride or a co-precipitate thereof within a capsule which may be coated with gelatin. Tablets and powders may also be coated with an enteric coating. Suitable enteric coating agents include phthalic acid cellulose acetate, hydroxypropylmethyl cellulose phthalate, polyvinyl alcohol phthalate, carboxy methyl ethyl cellulose, a copolymer of styrene and maleic acid, a copolymer of methacrylic acid and methyl methacrylate, and like materials, and if desired, the coating agents may be employed with suitable plasticizers and/or extending agents. A coated capsule or tablet may have a coating on the surface thereof or may be a capsule or tablet comprising a powder or granules with an enteric-coating.

Tableting compositions may have few or many components depending upon the tableting method used, the release rate desired and other factors. For example, the compositions described herein may contain diluents such as cellulose-derived materials like powdered cellulose, microcrystalline cellulose, micro fine cellulose, methyl cellulose, ethyl cellulose, hydroxyethyl cellulose, hydroxypropyl cellulose, hydroxypropylmethyl cellulose, carboxymethyl cellulose salts and other substituted and unsubstituted celluloses; starch; pregelatinized starch; inorganic diluents such calcium carbonate and calcium diphosphate and other diluents known to one of ordinary skill in the art. Yet other suitable diluents include waxes, sugars (e.g. lactose) and sugar alcohols such as mannitol and sorbitol, acrylate polymers and copolymers, as well as pectin, dextrin and gelatin.

Other excipients include binders, such as acacia gum, pregelatinized starch, sodium alginate, glucose and other binders used in wet and dry granulation and direct compression tableting processes; disintegrants such as sodium starch glycolate, crospovidone, low-substituted hydroxypropyl cellulose and others; lubricants like magnesium and calcium stearate and sodium stearyl fumarate; flavorings; sweeteners; preservatives; pharmaceutically acceptable dyes and glidants such as silicon dioxide.

### Instrumental Details:

### X-ray Powder Diffractometer:

The X-Ray powder diffraction was measured by an X-ray powder diffractometer equipped with a Cu-anode (k=1.54 Angstrom), X-ray source operated at 40 kV, 40 mA and a Ni filter is used to strip K-beta radiation. Two-theta calibration was performed using an NIST SRM 1976, Corundum standard. The sample was analyzed using the following instrument parameters: measuring range=3-45° 2-theta; step width=0.01579°; and measuring time per step=0.11 second.

The following examples are given for the purpose of illustrating the present disclosure and should not be considered as limitation on the scope or spirit of the disclosure.

### EXAMPLES

### Example 1

### Preparation of Amorphous Tapentadol Hydrochloride

Tapentadol hydrochloride (1 g) was dissolved in water (10 ml) and stirred for 20-30 minutes. The solution was filtered through a 0.45 micron filter, followed by addition of water and distillation using a Buchi evaporator at 70-75° C. under vacuum for 4-5 hours. The resulting mass was cooled at 20-25° C. to produce amorphous tapentadol hydrochloride (Yield: 85%; Moisture Content: 0.35% by weight).

### Example 2

### Preparation of Amorphous Tapentadol Hydrochloride

Tapentadol hydrochloride (1 g) was dissolved in water (10 ml) and stirred for 20-30 minutes. The solution was filtered through a 0.45 micron filter, and the filtrate was subjected to lyophilized distillation at −10° C. to −15° C. under 0.1 bar vacuum for 24-28 hours. The amorphous tapentadol hydrochloride was isolated under nitrogen atmosphere (Yield: 80%).

### Example 3

### Preparation of Amorphous Tapentadol Hydrochloride

Tapentadol hydrochloride (1 g) was dissolved in methanol (20 ml) and stirred for 20-30 minutes. The solution was filtered through a 0.45 micron filter, and the filtrate was subjected to spray drying for 2 hours under the conditions of inlet temperature at 81° C., outlet temperature at 48° C., aspirator 40 and feed pump rate 15%. The amorphous tapentadol hydrochloride was isolated under nitrogen atmosphere. (Yield: 80%).

### Example 4

### Preparation of Amorphous Coprecipitate of Tapentadol Hydrochloride with Povidone in a Ratio of 1:1.

Tapentadol hydrochloride (0.5 g) and povidone (0.5 g) were dissolved in methanol (20 ml) and stirred for 20-30 minutes. The solution was filtered through a 0.45 micron filter, and the solvent was distilled using a Buchi evaporator at 50-55° C. under vacuum for 4-5 hours. The resulting mass was cooled to 20-25° C. to produce amorphous coprecipitate of tapentadol hydrochloride with povidone in a ratio of 1:1 (Yield: 80%).

Unless otherwise indicated, the following definitions are set forth to illustrate and define the meaning and scope of the various terms used to describe the invention herein.

The term “amorphous” means a solid without long-range crystalline order. An amorphous form of tapentadol hydrochloride specifically contains less than about 10 percent crystalline forms of tapentadol hydrochloride, more specifically less than 5 percent crystalline forms of tapentadol hydrochloride, and still more specifically is essentially free of crystalline forms of tapentadol hydrochloride. “Essentially free of crystalline forms of tapentadol hydrochloride” means that no crystalline polymorph forms of tapentadol hydrochloride can be detected within the limits of a powder X-ray diffractometer.

The term “pharmaceutically acceptable” means that which is useful in preparing a pharmaceutical composition that is generally non-toxic and is not biologically undesirable, and includes that which is acceptable for veterinary use and/or human pharmaceutical use.

The term “pharmaceutical composition” is intended to encompass a drug product including the active ingredient(s), pharmaceutically acceptable excipients that make up the carrier, as well as any product which results, directly or indirectly, from combination, complexation or aggregation of any two or more of the ingredients. Accordingly, the pharmaceutical compositions encompass any composition made by admixing the active ingredient, active ingredient dispersion or composite, additional active ingredient(s), and pharmaceutically acceptable excipients.

The term “therapeutically effective amount” as used herein means the amount of a compound that, when administered to a mammal for treating a state, disorder or condition, is sufficient to effect such treatment. The “therapeutically effective amount” will vary depending on the compound, the disease and its severity and the age, weight, physical condition and responsiveness of the mammal to be treated.

The term “delivering” as used herein means providing a therapeutically effective amount of an active ingredient to a particular location within a host causing a therapeutically effective blood concentration of the active ingredient at the particular location. This can be accomplished, e.g., by topical, local or by systemic administration of the active ingredient to the host.

The term “buffering agent” as used herein is intended to mean a compound used to resist a change in pH upon dilution or addition of acid of alkali. Such compounds include, by way of example and without limitation, potassium metaphosphate, potassium phosphate, monobasic sodium acetate and sodium citrate anhydrous and dehydrate and other such materials known to those of ordinary skill in the art.

The term “sweetening agent” as used herein is intended to mean a compound used to impart sweetness to a formulation. Such compounds include, by way of example and without limitation, aspartame, dextrose, glycerin, mannitol, saccharin sodium, sorbitol, sucrose, fructose and other such materials known to those of ordinary skill in the art.

The term “binders” as used herein is intended to mean substances used to cause adhesion of powder particles in granulations. Such compounds include, by way of example and without limitation, acacia, alginic acid, tragacanth, carboxymethylcellulose sodium, polyvinylpyrrolidone, compressible sugar (e.g., NuTab), ethylcellulose, gelatin, liquid glucose, methylcellulose, pregelatinized starch, starch, polyethylene glycol, guar gum, polysaccharide, bentonites, sugars, invert sugars, poloxamers (PLURONIC™ F68, PLURONIC™ F127), collagen, albumin, celluloses in non-aqueous solvents, polypropylene glycol, polyoxyethylene-polypropylene copolymer, polyethylene ester, polyethylene sorbitan ester, polyethylene oxide, microcrystalline cellulose, combinations thereof and other material known to those of ordinary skill in the art.

The term “diluent” or “filler” as used herein is intended to mean inert substances used as fillers to create the desired bulk, flow properties, and compression characteristics in the preparation of solid dosage formulations. Such compounds include, by way of example and without limitation, dibasic calcium phosphate, kaolin, sucrose, mannitol, microcrystalline cellulose, powdered cellulose, precipitated calcium carbonate, sorbitol, starch, combinations thereof and other such materials known to those of ordinary skill in the art.

The term “glidant” as used herein is intended to mean agents used in solid dosage formulations to improve flow-properties during tablet compression and to produce an anti-caking effect. Such compounds include, by way of example and without limitation, colloidal silica, calcium silicate, magnesium silicate, silicon hydrogel, cornstarch, talc, combinations thereof and other such materials known to those of ordinary skill in the art.

The term “lubricant” as used herein is intended to mean substances used in solid dosage formulations to reduce friction during compression of the solid dosage. Such compounds include, by way of example and without limitation, calcium stearate, magnesium stearate, mineral oil, stearic acid, zinc stearate, combinations thereof and other such materials known to those of ordinary skill in the art.

The term “disintegrant” as used herein is intended to mean a compound used in solid dosage formulations to promote the disruption of the solid mass into smaller particles which are more readily dispersed or dissolved. Exemplary disintegrants include, by way of example and without limitation, starches such as corn starch, potato starch, pregelatinized, sweeteners, clays, such as bentonite, microcrystalline cellulose (e.g., Avicel™), carsium (e.g., Amberlite™), alginates, sodium starch glycolate, gums such as agar, guar, locust bean, karaya, pectin, tragacanth, combinations thereof and other such materials known to those of ordinary skill in the art.

The term “wetting agent” as used herein is intended to mean a compound used to aid in attaining intimate contact between solid particles and liquids. Exemplary wetting agents include, by way of example and without limitation, gelatin, casein, lecithin (phosphatides), gum acacia, cholesterol, tragacanth, stearic acid, benzalkonium chloride, calcium stearate, glycerol

monostearate, cetostearyl alcohol, cetomacrogol emulsifying wax, sorbitan esters, polyoxyethylene alkyl ethers (e.g., macrogol ethers such as cetomacrogol 1000), polyoxyethylene castor oil derivatives, polyoxyethylene sorbitan fatty acid esters, (e.g., TWEEN™s), polyethylene glycols, polyoxyethylene stearates colloidal silicon dioxide, phosphates, sodium dodecylsulfate, carboxymethylcellulose calcium, carboxymethylcellulose sodium, methylcellulose, hydroxyethylcellulose, hydroxylpropylcellulose, hydroxypropylmethylcellulose phthalate, noncrystalline cellulose, magnesium aluminum silicate, triethanolamine, polyvinyl alcohol, and polyvinylpyrrolidone (PVP).

The term “micronization” used herein means a process or method by which the size of a population of particles is reduced.

As used herein, the term “micron” or “μm” both are equivalent and refer to “micrometer” which is 1×10⁻⁶ meter.

As used herein, “crystalline particles” means any combination of single crystals, aggregates and agglomerates.

As used herein, “Particle Size Distribution (P.S.D)” means the cumulative volume size distribution of equivalent spherical diameters as determined by laser diffraction in Malvern Master Sizer 2000 equipment or its equivalent.

The important characteristics of the PSD are the (D₉₀), which is the size, in microns, below which 90% of the particles by volume are found, and the (D₅₀), which is the size, in microns, below which 50% of the particles by volume are found. Thus, a D₉₀ or d(0.9) of less than 300 microns means that 90 volume-percent of the particles in a composition have a diameter less than 300 microns.

The term “coprecipitate or co-precipitate” as used herein refers to compositions comprising amorphous tapentadol hydrochloride together with at least one pharmaceutically acceptable excipient, being prepared by removing solvent from a solution containing both of them.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

We claim:

1. An amorphous form of tapentadol hydrochloride or an amorphous co-precipitate comprising tapentadol hydrochloride and a pharmaceutically acceptable excipient, wherein the pharmaceutically acceptable excipient is selected from the group consisting of polyvinylpyrrolidone, polyvinyl alcohol, hydroxypropyl methylcellulose, methyl cellulose, carboxymethyl cellulose, sodium carboxymethyl cellulose, hydroxyethylcellulose, polyvinyl acetate, maltodextrins, cyclodextrins, gelatins, hypromellose phthalate, sugars, and combinations comprising one or more of the foregoing excipients.

2. The amorphous tapentadol hydrochloride of claim 1, wherein the pharmaceutically acceptable excipient is polyvinylpyrrolidone.

3. The amorphous tapentadol hydrochloride of claim 1, wherein the amorphous form of tapentadol hydrochloride is characterized by a powder X-ray diffraction pattern, showing a plain halo with no well-defined peaks, substantially in accordance with FIG. 1; and wherein the amorphous co-precipitate of tapentadol hydrochloride with polyvinylpyrrolidone is characterized by a powder X-ray diffraction pattern, showing a plain halo with no well-defined peaks, substantially in accordance with FIG. 2.

4. The amorphous tapentadol hydrochloride of claim 1, wherein the amorphous form of tapentadol hydrochloride or a co-precipitate thereof contains less than about 10 percent crystalline forms of tapentadol hydrochloride.

5. The amorphous tapentadol hydrochloride of claim 4, wherein the amorphous form of tapentadol hydrochloride or a co-precipitate thereof contains less than about 5 percent crystalline forms of tapentadol hydrochloride.

6. The amorphous tapentadol hydrochloride of claim 5, wherein the amorphous form of tapentadol hydrochloride or a co-precipitate thereof contains less than about 1 percent crystalline forms of tapentadol hydrochloride.

7. A process for the preparation of the amorphous tapentadol hydrochloride of claim 1, comprising:
a) providing a solution of tapentadol hydrochloride in a solvent, wherein the solvent is water, an organic solvent, or a solvent medium comprising water and an organic solvent, and wherein the organic solvent is selected from the group consisting of an alcohol, a ketone, a chlorinated hydrocarbon, a hydrocarbon, a C₁-C₃ carboxylic acid, a nitrile, a polar aprotic solvent, and mixtures thereof;
b) optionally, filtering the solution to remove insoluble matter;
c) optionally, seeding the solution; and
d) substantially removing the solvent from the solution to provide the amorphous form of tapentadol hydrochloride.

8. The process of claim 7, wherein the organic solvent used in step-(a) is selected from the group consisting of methanol, ethanol, n-propanol, isopropyl alcohol, isobutanol, n-butanol, tert-butanol, acetone, methyl ethyl ketone, methyl isobutyl ketone, methyl tert-butyl ketone, methylene chloride, ethylene dichloride, chloroform, n-pentane, n-hexane, n-heptane, cyclohexane, toluene, xylene, acetic acid, acetonitrile, N,N-dimethylformamide, N,N-dimethylacetamide, dimethylsulfoxide, and mixtures thereof; and wherein the solution in step-(a) is provided either i) by dissolving the tapentadol hydrochloride in the solvent at a temperature of below about reflux temperature of the solvent; or ii) by admixing tapentadol base, hydrochloric acid and the solvent to obtain a mixture; and stirring the mixture to obtain a solution of tapentadol hydrochloride.

9. The process of claim 8, wherein the organic solvent is methanol; and wherein the dissolution of tapentadol hydrochloride in the solvent is carried out at a temperature of about 20° C. to about 110° C.

10. The process of claim 7, wherein the solution obtained in step-(a) is optionally subjected to carbon treatment or silica gel treatment; wherein the solution obtained in step-(a) is optionally stirred at a temperature of about 30° C. to the reflux temperature of the solvent used for at least 20 minutes; wherein the removal of the solvent in step-(d) is accomplished by distillation or complete evaporation of the solvent, spray drying, vacuum drying, lyophilization or freeze drying, agitated thin-film (ATFD) drying, or a combination thereof; wherein the amorphous tapentadol hydrochloride obtained in step-(d) is further dried under vacuum or at atmospheric pressure, at a temperature of about 25° C. to about 70° C.; and wherein the amorphous tapentadol hydrochloride obtained has a purity of about 99% to about 99.99% as measured by HPLC.

11. A process for the preparation of an amorphous co-precipitate of tapentadol hydrochloride and a pharmaceutically acceptable excipient of claim 1, comprising:
a) providing a solution of tapentadol hydrochloride and the pharmaceutically acceptable excipient in a solvent, wherein the solvent is water, an organic solvent, or a solvent medium comprising water and an organic solvent, and wherein the organic solvent is selected from the group consisting of an alcohol, a ketone, a chlorinated hydrocarbon, a hydrocarbon, a C₁-C₃ carboxylic acid, a nitrile, a polar aprotic solvent, and mixtures thereof;
b) optionally, filtering the solvent solution to remove insoluble matter; and
c) substantially removing the solvent from the solution to afford the amorphous co-precipitate of tapentadol hydrochloride with the pharmaceutically acceptable excipient.

12. The process of claim 11, wherein the organic solvent used in step-(a) is selected from the group consisting of methanol, ethanol, n-propanol, isopropyl alcohol, isobutanol, n-butanol, tert-butanol, acetone, methyl ethyl ketone, methyl isobutyl ketone, methyl tert-butyl ketone, methylene chloride, ethylene dichloride, chloroform, n-pentane, n-hexane, n-heptane, cyclohexane, toluene, xylene, acetic acid, acetonitrile, N,N-dimethylformamide, N,N-dimethylacetamide, dimethylsulfoxide, and mixtures thereof; and wherein the pharmaceutically acceptable excipient is selected from the group consisting of polyvinylpyrrolidone, polyvinyl alcohol, hydroxypropyl methylcellulose, methyl cellulose, carboxymethyl cellulose, sodium carboxymethyl cellulose, hydro xyethylcellulose, polyvinyl acetate, maltodextrins, cyclo dextrins, gelatins, hypromellose phthalate, sugars, and combinations comprising one or more of the foregoing excipients.

13. The process of claim 12, wherein the organic solvent is methanol; and wherein the pharmaceutically acceptable excipient is polyvinylpyrrolidone.

14. The process of claim 11, wherein the solution in step-(a) is provided either i) by dissolving tapentadol hydrochloride in the solvent and followed by combining the solution with the pharmaceutical excipient; or ii) by dissolving the pharmaceutical excipient in the solvent and followed by combining the solution with tapentadol hydrochloride; or iii) by combining the solution containing tapentadol hydrochloride with a solution containing the pharmaceutically acceptable excipient.

15. The process of claim 11, wherein the solution obtained in step-(a) is optionally subjected to carbon treatment or silica gel treatment; and wherein the removal of the solvent in step-(c) is accomplished by distillation or complete evaporation of the solvent, spray drying, vacuum drying, lyophilization or freeze drying, agitated thin-film drying, or a combination thereof.

16. A pharmaceutical composition comprising amorphous tapentadol hydrochloride or an amorphous co-precipitate comprising tapentadol hydrochloride and a pharmaceutically acceptable excipient of claim 1, and one or more second pharmaceutically acceptable excipients.

17. The pharmaceutical composition of claim 16, wherein the pharmaceutical composition is a solid dosage form, an oral suspension, a liquid, a powder, an elixir, an aerosol, syrups or an injectable solution.

18. The pharmaceutical composition of claim 16, wherein the amorphous tapentadol hydrochloride or a co-precipitate thereof has a D₉₀ particle size of less than or equal to about 500 microns.

19. The pharmaceutical composition of claim 18, wherein the D₉₀ particle size is about 1 micron to about 300 microns, or about 10 microns to about 150 microns.

20. A method for treating a patient suffering from severe acute pain, comprising administering a therapeutically effective amount of an amorphous tapentadol hydrochloride or an amorphous co-precipitate comprising tapentadol hydrochloride and a pharmaceutically acceptable excipient, or a pharmaceutical composition that comprises a therapeutically effective amount of amorphous tapentadol hydrochloride or an amorphous co-precipitate comprising tapentadol hydrochloride and a pharmaceutically acceptable excipient, along with a second pharmaceutically acceptable excipient.
